(19) United States
(12) Patent Application Publication
(10) Pub. No.: US 2020/0365263 A1
(43) Pub. Date: Nov. 19, 2020

(54) INFORMATION PROCESSING DEVICE, MOVING VEHICLE, AND INFORMATION PROCESSING METHOD

(71) Applicant: TOYOTA JIDOSHA KABUSHIKI KAISHA, Toyota-shi (JP)

(72) Inventors: Atsuko KOBAYASHI, Nagoya-shi (JP); Kenji FUJIHARA, Nagoya-shi (JP); Akitoshi JIKUMARU, Nisshin-shi (JP); Mizuki KIUCHI, Toyota-shi (JP); Takashi YAMAZAKI, Nagoya-shi (JP); Masayuki GOTO, Nagoya-shi (JP)

(73) Assignee: TOYOTA JIDOSHA KABUSHIKI KAISHA, Toyota-shi (JP)

(21) Appl. No.: 16/827,771

(22) Filed: Mar. 24, 2020

(30) Foreign Application Priority Data
May 13, 2019 (JP) 2019-090773

(51) Int. Cl. G16H 40/60; G16H 40/20; G16H 10/60; G01C 21/20; B60W 60/00

(52) U.S. Cl. CPC G16H 40/60; G16H 40/20; B60W 60/0016; G01C 21/206; B60W 60/0011; G16H 10/60

(57) ABSTRACT

An information processing device manages the running of a moving vehicle that contains a bed on which an examinee is laid and that can run autonomously. The information processing device is equipped with a control unit that carries out acquisition of an exam schedule of the examinee, generation of a movement instruction signal for moving the moving vehicle to a place of exam, based on the exam schedule, and transmission of the movement instruction signal to the moving vehicle.

### INCORPORATION BY REFERENCE

The disclosure of Japanese Patent Application No. 2019-090773 filed on May 13, 2019 including the specification, drawings and abstract is incorporated herein by reference in its entirety.

### BACKGROUND

### 1. Technical Field

The disclosure relates to an information processing device that manages the running of a moving vehicle that contains a bed on which an examinee is laid, the moving vehicle, and an information processing method.

### 2. Description of Related Art

In Japanese Patent Application Publication No. 09-183334 (JP 09-183334 A), there is proposed a mobile office that has pieces of office equipment arranged usably in a vehicle and that allows paperwork to be done immediately at a destination without laying out the pieces of office equipment.

### SUMMARY

It is an object of the disclosure to provide an art capable of improving the user-friendliness that is offered when an examinee is examined.

An information processing device according to a first aspect of the disclosure manages the running of a moving vehicle that contains a bed on which an examinee is laid, and that can run autonomously. The information processing device is equipped with a control unit that carries out acquisition of an exam schedule of the examinee, generation of a movement instruction signal for moving the moving vehicle to a place of exam, based on the exam schedule, and transmission of the movement instruction signal to the moving vehicle.

A moving vehicle according to a second aspect of the disclosure contains a bed on which an examinee is laid, and can run autonomously. The moving vehicle is equipped with a drive unit for causing the moving vehicle to run, and a control unit that controls the drive unit. The control unit carries out reception of a movement instruction signal generated based on an exam schedule of the examinee, from another information processing device, and control of the drive unit based on the movement instruction signal to cause the moving vehicle to autonomously run to a place of exam.

An information processing method according to a third aspect of the disclosure is carried out by an information processing device that manages the running of a moving vehicle that contains a bed on which an examinee is laid and that can run autonomously. The information processing method includes a step of acquiring an exam schedule of the examinee, a step of generating a movement instruction signal based on the exam schedule, and a step of transmitting the movement instruction signal to the moving vehicle.

The disclosure can improve the user-friendliness that is offered when the examinee is examined.

### BRIEF DESCRIPTION OF THE DRAWINGS

Features, advantages, and technical and industrial significance of exemplary embodiments will be described below with reference to the accompanying drawings, in which like signs denote like elements, and wherein:

FIG. 1 is a view showing a schematic configuration of a moving vehicle system according to one of the embodiments;

FIG. 2 is a block diagram schematically showing an example of a functional configuration of a moving vehicle and a moving vehicle management server that constitute the moving vehicle system according to the embodiment;

FIG. 3 is a view showing an example of a data configuration of an exam schedule that is stored in an exam schedule database;

FIG. 4 is a view showing an example of a data configuration of an adjustment data table;

FIG. 5 is a view showing the flow of a movement instruction process that is performed by the movement vehicle management server;

FIG. 6 is a view showing the flow of a movement process that is performed by the moving vehicle; and

FIG. 7 is a view showing an example of a moving vehicle according to a modification example of the embodiment.

### DETAILED DESCRIPTION OF EMBODIMENTS

An information processing device according to the disclosure manages the running of a moving vehicle that contains a bed on which an examinee is laid, and that can run autonomously. The information processing device generates a movement instruction signal for moving the moving vehicle to a place of exam, based on an exam schedule of the examinee, and transmits this movement instruction signal to the moving vehicle. By receiving this movement instruction signal from the information processing device, the moving vehicle moves through autonomous running, based on the movement instruction signal. Thus, the information processing device can move the moving vehicle in which the examinee rides, in accordance with the exam schedule, and can improve the user-friendliness that is offered when the examinee is examined.

It should be noted herein that the exam is a medical practice of asking a subject questions and examining the body thereof with a view to diagnosing the medical condition thereof in, for example, a medical institution. Besides, in the present embodiment, procedures for diagnosis and procedures resulting from diagnosis, such as inspection, treatment, medication, operation, guidance, follow-up, counseling, rehabilitation, and the like, as well as asking questions and examining the body are each referred to as an exam. The exam schedule is a schedule of such an exam. The exam schedule can have information on the date and hour of exam, the type of exam, the place of exam, and the like.

One of the concrete embodiments will be described hereinafter based on the drawings. The dimensions, materials, shapes, relative arrangement, and the like of components mentioned in the present embodiment are not intended to limit the technical scope of the disclosure thereto, unless otherwise specified.

### Embodiment

### Outline of System

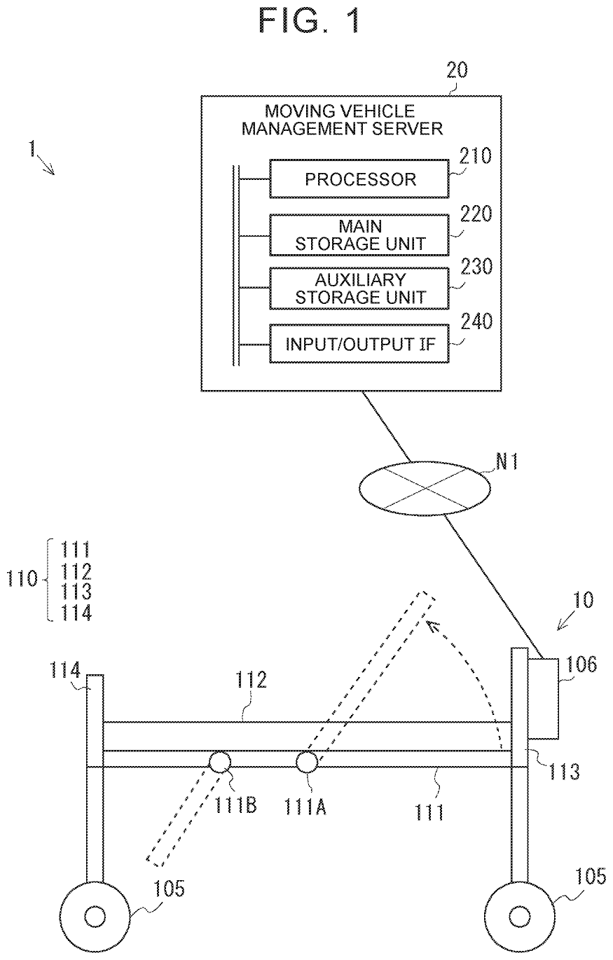

FIG. 1 is a view showing the schematic configuration of a moving vehicle system according to the present embodiment. A moving vehicle system 1 is configured to include a moving vehicle 10 capable of running autonomously, and a moving vehicle management server 20. In the moving vehicle system 1, the moving vehicle 10 and the moving vehicle management server 20 are connected to each other by a communication line N1. For example, a public communication network such as the Internet or the like, Wide Area Network (WAN), Local Area Network (LAN), or a cellular phone line may be adopted as the communication line N1.

The moving vehicle 10 according to the present embodiment is provided in a patient's room or the like in a hospital, and has a drive unit 105 for running in the hospital, a control unit 106 that controls the drive unit 105, and a bed 110 on which an examinee is laid. When the control unit 106 receives a movement instruction signal that is transmitted from the moving vehicle management server 20, via the communication line N1, and controls the drive unit 105 based on the movement instruction signal, the moving vehicle 10 can thereby move to a place of exam through autonomous running.

The bed 110 has a mattress 112 on a floorboard 111. In the case where the examinee lies on the mattress 112, the bed 110 has a headboard 113 on a head side of the examinee, and has a footboard 114 on a foot side of the examinee. The floorboard 111 can turn the head side upward, around a turning shaft 111A provided at a middle portion between the head side and the foot side. Besides, the floorboard 111 can turn the foot side downward, around a turning shaft 111B provided closer to the foot side than the turning shaft 111A. That is, the examinee can be placed in a chair-like state, with the head side of each of the floorboard 111 and the mattress 112 raised, and with toes of the examinee lowered. The moving vehicle 10 can move in this sitting mode, or in a lying mode in which the floorboard 111 and the mattress 112 are horizontal.

The moving vehicle management server 20 is configured to include a general computer. The computer constituting the moving vehicle management server 20 is equipped with a processor 210 such as a CPU, a DSP, or the like, a main storage unit 220 such as a read-only memory (ROM), a random access memory (RAM), or the like, an auxiliary storage unit 230 such as an EPROM, a hard disk drive (HDD), a removable medium, or the like, and an input/output interface (IF) 240. It should be noted herein that the processor 210 performs control and the like of the entire moving vehicle management server 20, by processing input information, and outputting a processing result. The processor 210 is also referred to as a Central Processing Unit (CPU) or a Micro-Processing Unit (MPU). The processor 210 may not necessarily be a single processor, but may have a multi-processor configuration. Alternatively, the processor 210 may have a multi-core configuration having a plurality of cores in a single chip that is connected by a single socket. The removable medium is, for example, a flash memory such as a USB memory, an SD card, or the like, or a disk recording medium such as a CD-ROM, a DVD disk, or a Blu-ray disk.

In the moving vehicle management server 20, an operating system (OS), various programs, various information tables, and the like are stored in the auxiliary storage unit 230. Besides, when the processor 210 loads the programs (software) stored in the auxiliary storage unit 230 into the main storage unit 220 and executes the programs, various functional units as will be described later can thereby be realized in the moving vehicle management server 20. It should be noted, however, that one, some, or all of the respective functional units in the moving vehicle management server 20 may be formed by a hardware circuit such as a dedicated Large-Scale Integration (LSI) such as a digital signal processor (DSP), an application specific integrated circuit (ASIC), a field-programmable gate array (FPGA) etc., a logic circuit, or another digital circuit. Besides, at least one or some of the aforementioned respective functional units may be configured to include an analog circuit. Incidentally, the moving vehicle management server 20 may not necessarily be realized by a single physical configuration, but may be constituted by a plurality of computers that operate in coordination with one another.

The input/output IF 240 is an interface to/from which data are input/output from/to pieces of equipment that are connected to the moving vehicle management server 20. Data are input/output to/from, for example, pieces of equipment such as an operation unit, a disk drive that reads data from a storage medium such as a CD, a DVD, or the like, a card reader/writer, a display device, and the like from/to the input/output IF 240. The operation unit is an input unit to which information for the moving vehicle management server 20 is input through operation of an input button, a keyboard, a pointing device, a touch panel, or the like by a user. For example, a schedule of the examinee and the like are input to the input unit.

The moving vehicle management server 20 is connected to a plurality of moving vehicles 10, and controls the running of the respective moving vehicles 10. Therefore, the moving vehicle management server 20 acquires an exam schedule of an examinee riding in each of the moving vehicles 10. For example, a medical staff including doctors, nurses, laboratory technicians, physiotherapists, and the like read out exam schedules registered in the moving vehicle management server 20, and acquire the exam schedules. Besides, the medical staff may acquire information such as reservations for exams recorded on electronic medical records, instructions on medication, instructions on rehabilitation, and the like, as the exam schedules, from a medical record management server 30 that will be described later.

### System Configuration

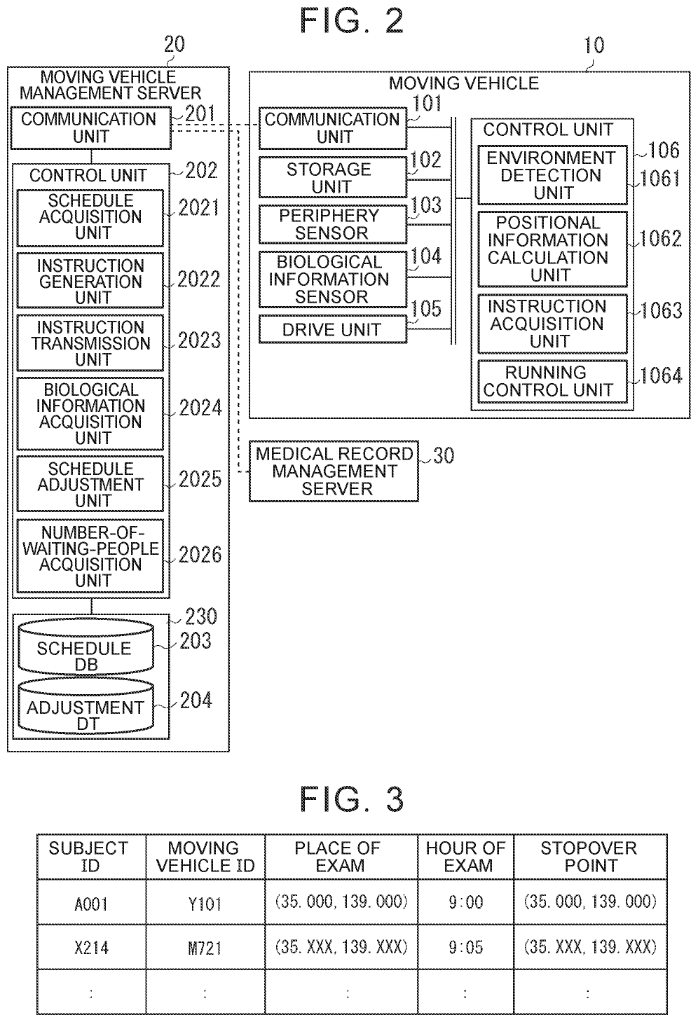

Next, the functional configuration of each of the moving vehicle 10 and the moving vehicle management server 20 according to the present embodiment will be described based on FIG. 2. FIG. 2 is a block diagram schematically showing an example of the functional configuration of each of the moving vehicle 10 and the moving vehicle management server 20 that constitute the moving vehicle system 1 according to the present embodiment.

### Autonomous Moving vehicle

As described above, the moving vehicle 10 is an autonomous moving vehicle that autonomously runs based on a movement instruction signal that is transmitted from the moving vehicle management server 20. The moving vehicle 10 is configured to include a communication unit 101, a storage unit 102, a periphery sensor 103, a biological information sensor 104, a drive unit 105, a control unit 106, and a bed 110. Incidentally, the moving vehicle 10 is an electrically operated moving vehicle having a motor that serves as a prime mover.

The communication unit 101 is a communication device for connecting the moving vehicle 10 to the communication line N1. The communication unit 101 can communicate with other devices including the moving vehicle management server 20, via the communication line N1, through the use of, for example, a moving vehicle communication service such as 3rd generation (3G), long-term evolution (LTE), or the like. The control unit 106 performs a process of receiving a movement instruction signal from the moving vehicle management server 20 via the communication unit 101 as will be described later. Besides, the control unit 106 performs a process of controlling the drive unit 105 based on the movement instruction signal.

The storage unit 102 is a device that stores information, and is constituted by a storage medium such as a magnetic disk, a flash memory, or the like. For example, map information is stored in the storage unit 102. Besides, the movement instruction signal received from the moving vehicle management server 20 is stored into the storage unit 102.

The periphery sensor 103 is a device for sensing a situation around the moving vehicle 10. In concrete terms, the periphery sensor 103 is configured to include a stereo camera, a laser scanner, a LIDAR, a radar, an acceleration sensor, an azimuth sensor, a speed sensor, an odometer, a GPS receiver, or the like. Information on the situation around the moving vehicle 10 that has been acquired by the periphery sensor 103 is transmitted to the control unit 106.

The biological information sensor 104 is a sensor that acquires biological information on the examinee riding in the moving vehicle 10. For example, a clinical thermometer, an electrocardiographic monitor, a pulse oximeter (a blood oxygen saturation level meter), or a blood pressure gauge can be mentioned as the biological information sensor 104. The biological information on the examinee that has been detected by the biological information sensor 104 is transmitted to the moving vehicle management server 20.

The control unit 106 has a function of performing a computation process for controlling the moving vehicle 10. The control unit 106 is constituted by, for example, a microcomputer. The control unit 106 has, as functional modules thereof, an environment detection unit 1061, a positional information calculation unit 1062, an instruction acquisition unit 1063, and a running control unit 1064. The respective functional modules may be realized through the execution of the programs stored in the storage unit such as the ROM or the like belonging to the control unit 106, by the processor such as the CPU or the like. Alternatively, one, some, or all of the functions may be realized by a hardware circuit such as an ASIC or an FPGA.

The environment detection unit 1061 detects an environment around the moving vehicle 10, based on the information acquired by the periphery sensor 103. For example, the environment detection unit 1061 detects objects (including human beings and animals) such as other moving vehicles and the like that are present around the environment detection unit 1061. Besides, the environment detection unit 1061 detects various subject matters that are needed for autonomous running of the moving vehicle 10, such as signs and the like in the hospital. Besides, the environment detection unit 1061 may track the detected objects. In this case, for example, a relative speed of each of the objects may be obtained from a difference between a coordinate of the object detected one step prior to the current time and the current coordinate of the object.

The positional information calculation unit 1062 acquires a current position of the moving vehicle 10, based on the information acquired by the periphery sensor 103, the communication unit 101, and the like. For example, a position-indicating sign that is provided on a passageway or the like is photographed by a camera, and a position of the sign is identified from positional information indicated by the sign, referring to the photographed image. Then, the positional information calculation unit 1062 detects relative positional information such as a moving direction, a moving distance, and the like of the moving vehicle 10, with respect to the position of the sign, by the periphery sensor 103, and obtains a position of the moving vehicle 10 according to autonomous navigation, through the use of an absolute position of the sign and the relative position detected by the periphery sensor 103. Besides, upon receiving a beacon signal from an access point where the communication unit 101 establishes wireless communication, the positional information calculation unit 1062 recognizes that the moving vehicle is located near this access point, and obtains the position of the moving vehicle 10 according to autonomous navigation, with respect to the position of this access point, through the use of an absolute position of this access point and the relative position detected by the periphery sensor 103. Furthermore, in the case where a GPS signal can be received, the positional information calculation unit 1062 obtains the position of the moving vehicle, based on the GPS signal.

The instruction acquisition unit 1063 receives a movement instruction signal generated based on the exam schedule of the examinee, from the moving vehicle management server 20. It should be noted herein that the movement instruction signal has, for example, information indicating a place of exam as a destination and a stopover point. Incidentally, in the case where the place of exam is identified in advance, the movement instruction signal may not include the information on the place of exam. Besides, the movement instruction signal may include information indicating a running route, an hour of arrival, an hour of departure, and the like.

Besides, the running control unit 1064 controls the running of the moving vehicle 10, based on the received movement instruction signal, and data on the current position of the moving vehicle 10 calculated by the positional information calculation unit 1062 and a surrounding environment detected by the environment detection unit 1061. For example, the running control unit 1064 causes the moving vehicle 10 to run from the current position to the place of exam indicated by the movement instruction signal. It should be noted herein that the running control unit 1064 reads out map information in the hospital from the storage unit 102, and performs collision avoidance control for causing the moving vehicle 10 to run in such a manner as to avoid a collision with an object with which the moving vehicle 10 may collide, when the environment detection unit 1061 detects the object from the current position. Incidentally, a known method can be adopted as a method of causing the moving vehicle 10 to run autonomously as described above.

The drive unit 105 is configured to include a motor as a prime mover, and a mechanism (e.g., a brake) for causing the moving vehicle 10 to run. The drive unit 105 causes the moving vehicle 10 to run, based on a command generated by the running control unit 1064 to control the running of the moving vehicle 10. Thus, autonomous running of the moving vehicle 10 is realized.

### Management Server

The moving vehicle management server 20 is configured to include a communication unit 201, a control unit 202, an exam schedule database (an exam schedule DB) 203, and an adjustment data table (an adjustment DT) 204. The communication unit 201 is a communication device for connecting the moving vehicle management server 20 to the communication line N1. The communication unit 201 is configured to include, for example, a local area network (LAN) interface board, and a wireless communication circuit for wireless communication.

The control unit 202 generates a movement instruction signal for moving each of the moving vehicles 10 to the place of exam, based on the exam schedule of the examinee. Besides, the control unit 202 performs a process of transmitting the movement instruction signal to each of the moving vehicles 10 via the communication unit 201.

Besides, the control unit 202 performs a process of acquiring exam schedules of respective examinees, and storing the acquired exam schedules into the exam schedule DB 203. Incidentally, when biological information on the examinees fulfills a predetermined condition, for example, when the conditions of the examinees deteriorate, the control unit 202 changes the exam schedules with reference to the adjustment DT.

FIG. 3 is a view showing an example of a data configuration of the exam schedules stored in the exam schedule DB 203. As shown in FIG. 3, each of the exam schedules has a subject ID field, a moving vehicle ID field, an exam place field, an exam hour field, and a stopover point field. A subject ID as identification information for identifying an examinee is input to the subject ID field. A moving vehicle ID as identification information for identifying the moving vehicle 10 is input to the moving vehicle ID field. A destination of the moving vehicle 10, namely, positional information indicating a place where an exam is conducted is input to the exam place field. An hour when the exam is started is input to the exam hour field. A place where a stopover is made in moving to the place of exam, such as a waiting room for those who are to be examined or the like is input to the stopover point field. Incidentally, in the case where there is no place where a stopover is made, no information may be input to the stopover point field.

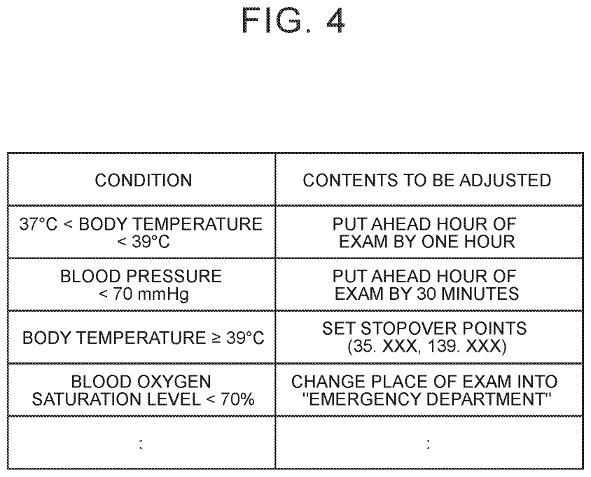

FIG. 4 is a view showing an example of a data configuration of the adjustment DT 204. As shown in FIG. 4, the adjustment DT 204 has a condition field and an adjustment content field. A condition on biological information that requires the adjustment of a schedule is input to the condition field. Information indicating the contents for adjusting an exam schedule in the case where the biological information fulfills the condition designated in the condition field is input to the adjustment field.

The control unit 202 has a function of performing a computation process for controlling the moving vehicle management server 20. The control unit 202 can be realized by the processor 210 and the main storage unit 220. Besides, the control unit 202 has, as functional modules thereof, a schedule acquisition unit 2021, an instruction generation unit 2022, an instruction transmission unit 2023, a biological information acquisition unit 2024, a schedule adjustment unit 2025, and a number-of-waiting-people acquisition unit 2026.

The schedule acquisition unit 2021 acquires exam schedules of examinees. For example, the medical staff read out the exam schedules registered in the moving vehicle management server 20 from the storage unit 230, and acquire the exam schedules. Besides, the schedule acquisition unit 2021 acquires, as the exam schedules, information such as reservations for exams recorded on electronic medical records by the medical staff, instructions on medication, instructions on rehabilitation, and the like, from the medical record management server 30.

The instruction generation unit 2022 generates a movement instruction signal for moving each of the moving vehicles 10 to the place of exam, based on the corresponding one of the exam schedules acquired by the schedule acquisition unit 2021.

The instruction transmission unit 2023 transmits the movement instruction signal generated by the instruction generation unit 2022, to each of the moving vehicles 10, via the communication line N1.

The biological information acquisition unit 2024 receives, from each of the moving vehicles 10, the biological information detected by the biological information sensor 104 of each of the moving vehicles 10.

The schedule adjustment unit 2025 adjusts the exam schedule when the biological information acquired by the biological information acquisition unit 2024 fulfills a predetermined condition. In concrete terms, when the acquired biological information fulfills the condition designated in the condition field of the adjustment DT, the schedule adjustment unit 2025 adjusts the exam schedule in accordance with the adjustment contents designated in the corresponding adjustment content field. For example, when the body temperature is higher than a first threshold (e.g., 37° C.) and lower than a second threshold (e.g., 39° C.), or when the blood pressure is lower than a third threshold (e.g., 70 mmHg), the hour of exam is put ahead by a predetermined time. Besides, when the body temperature is equal to or higher than the second threshold, a stopover is made in a waiting room for infectious diseases, the examinee is made to wait in the waiting room until the hour of exam, and the examinee is moved from the waiting room to an exam room as soon as the hour of exam comes. Furthermore, when the heart rate is lower than a fourth threshold, or when the blood oxygen saturation level is lower than a fifth threshold (70%), the place of exam is changed into “an emergency department”, and the examinee is immediately moved thereto.

The number-of-waiting-people acquisition unit 2026 acquires the number of other examinees waiting to be examined at the place of exam. For example, the number-of-waiting-people acquisition unit 2026 acquires, from the medical record management server 30, the medical schedules of the other examinees to be examined, obtains exam orders based on the hours of exam of the respective examinees, and calculates the number of examinees waiting to be examined earlier in order than the examinees selected to generate a movement instruction signal. When the number-of-waiting-people acquisition unit 2026 acquires the number of waiting people, the instruction generation unit 2022 generates a movement instruction signal based on this number of waiting people and the exam schedules. For example, the number-of-waiting-people acquisition unit 2026 obtains a waiting time until an exam, based on the number of waiting people, adjusts each of the hours of exam registered in each of the exam schedules, based on this waiting time, and generates a movement instruction signal. Besides, the number-of-waiting-people acquisition unit 2026 sets a departure time for movement through back calculation from this adjusted hour of exam, and generates a movement instruction signal including this departure time.

The medical record management server 30 is a computer that manages information on the electronic medical records. The medical record management server 30 is connected to the moving vehicle management server 20 via the communication line N1, and transmits the exam schedules in the electronic medical records to the moving vehicle management server 20.

### Movement Instruction Process

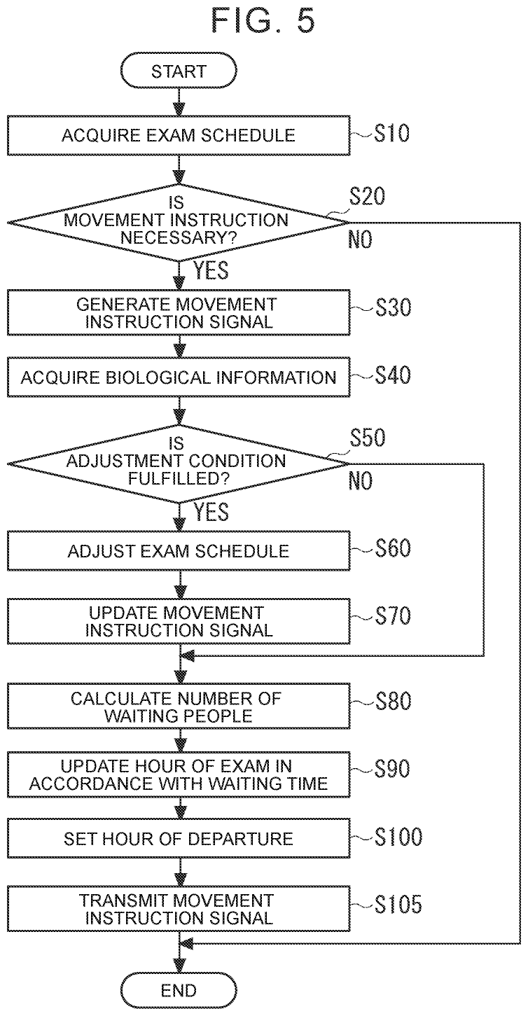

FIG. 5 is a view showing the flow of a movement instruction process that is performed by the moving vehicle management server 20. The moving vehicle management server 20 repeatedly performs the process of FIG. 5 on a predetermined cycle.

In step S10, the moving vehicle management server 20 selects an examinee based on a subject ID, and acquires an exam schedule of the examinee. Incidentally, FIG. 5 shows the process of issuing an instruction on the movement of the single examinee, but the moving vehicle management server 20 sequentially selects all the examinees waiting to be examined, and repeats the process of FIG. 5.

In step S20, the moving vehicle management server 20 determines whether or not a movement instruction is necessary as to the exam schedule acquired in step S10. For example, if there is a predetermined time or more before the hour and date of exam of the exam schedule, and the timing for issuing an instruction on movement has not come yet, no instruction is necessary, so the moving vehicle management server 20 makes a negative determination. Besides, the moving vehicle management server 20 also makes a negative determination when a movement instruction signal has already been transmitted as to the acquired exam schedule. On the other hand, if there is less than the predetermined time before the hour and date of exam of the acquired exam schedule, and no movement instruction signal has been transmitted yet, the moving vehicle management server 20 makes a positive determination.

If the result of the determination in step S20 is positive, the moving vehicle management server 20 shifts to step S30, and generates a movement instruction signal, based on the exam schedule acquired in step S10.

After step S30, the moving vehicle management server 20 shifts to step S40, and acquires biological information on the examinee from the moving vehicle 10.

In step S50, the moving vehicle management server 20 determines, with reference to the adjustment DT, whether or not the biological information acquired in step S40 fulfills a condition that requires adjustment.

If the result of the determination in step S50 is positive, the moving vehicle management server 20 shifts to step S60, and changes the exam schedule based on the adjustment DT. In concrete terms, the moving vehicle management server 20 carries out adjustments such as putting ahead the hour of exam, changing the place of exam, setting a stopover point, and the like.

In step S70, the moving vehicle management server 20 updates the movement instruction signal generated in step S30, based on the adjusted exam schedule.

After step S70, or if the result of the determination in step S50 is negative, the moving vehicle management server 20 shifts to step 80, obtains orders of examinees waiting to be examined with reference to the exam schedules of the respective examinees, and calculates the number of examinees who are earlier in order than the examinee selected in step S10, as the number of waiting people.

In step S90, the moving vehicle management server 20 obtains a waiting time by multiplying the number of waiting people obtained in step S80 by a standard time of exam, regards an hour after the lapse of the waiting time from the current hour, as an estimated hour of exam, and updates the hour of exam indicated by the movement instruction signal based on the estimated hour of exam, when the hour of exam of the exam schedule and the estimated hour of exam are different from each other by a predetermined threshold or more. Incidentally, it is not indispensable to update the hour of exam in consideration of the waiting time in the way described above.

In step S100, the moving vehicle management server 20 sets, as an hour of departure, an hour preceding the hour of exam included in the movement instruction signal by a predetermined time, and includes this hour of departure into the updated movement instruction signal. Then, in step S105, the moving vehicle management server 20 transmits the movement instruction signal to the moving vehicle 10 in which the examinee selected in step S10 rides. It should be noted herein that the predetermined time is determined as a standard time that is required for the movement to the place of exam. Incidentally, the moving vehicle management server 20 may acquire a current position from the moving vehicle 10, obtain a moving time in accordance with a moving distance from the current position to the place of exam, and set, as the hour of departure, an hour preceding the hour of exam included in the movement instruction signal by the moving time. Incidentally, the moving vehicle management server 20 may transmit the movement instruction signal to the moving vehicle 10 upon the arrival of the hour of departure, and then move the moving vehicle 10, instead of including the hour of exam and the hour of departure into the movement instruction signal.

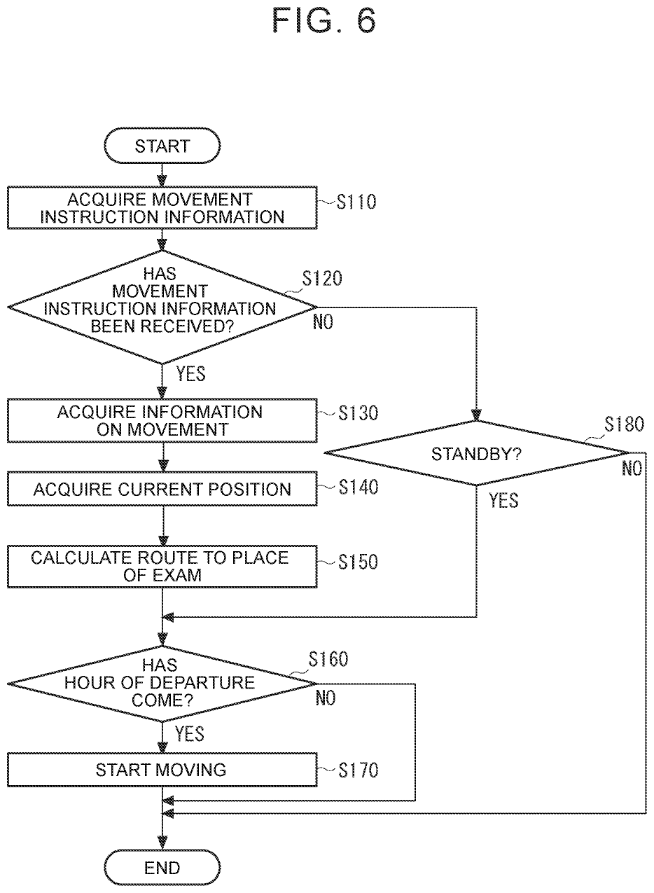

FIG. 6 is a view showing the flow of a movement process that is performed by the moving vehicle 10. The moving vehicle 10 repeatedly performs the process of FIG. 6 on a predetermined cycle.

In step S110, the moving vehicle 10 acquires a movement instruction signal from the moving vehicle management server 20.

In step S120, the moving vehicle 10 determines whether or not the movement instruction signal has been received in step S110.

If the result of the determination in step S120 is positive, the moving vehicle 10 shifts to step S130, and acquires information on movement such as a place of exam, an hour of departure (an hour of the start of movement), a stopover point, and the like as indicated by the movement instruction signal received in step S110. Incidentally, in the case where the movement instruction signal does not indicate information such as the place of exam, the hour of exam, or the like, and the movement to a predetermined place may be started upon reception of the movement instruction signal, the processing of step S120 can be omitted.

In step S140, the moving vehicle 10 acquires a current position. Besides, in step S150, the moving vehicle 10 reads out map information from the storage unit 102, and obtains a route from the current position to the place of exam. It should be noted herein that when a stopover point is designated, the moving vehicle 10 calculates a route passing through the stopover point.

In step S160, the moving vehicle 10 determines whether or not the hour of departure has come. If the result of the determination in step S160 is positive, the moving vehicle 10 shifts to step S170, and starts moving toward the place of exam based on the movement instruction signal.

On the other hand, if the result of the determination in step S160 is negative, namely, if the hour of departure has not arrived yet and the moving vehicle 10 stands by instead of starting moving, the moving vehicle 10 ends the process of FIG. 6.

Incidentally, if the result of the determination in step S160 is negative, a flag indicating that the moving vehicle 10 is in a standby state may be retained by the main storage unit or the like. The moving vehicle 10 periodically performs the process of FIG. 6. Therefore, even after having temporarily ended the process of FIG. 6 in step S160, the moving vehicle 10 performs the process again from step S110 on a subsequent cycle. At this time, when new movement instruction information is acquired in step S110, for example, when the exam schedule is changed due to a deterioration in the condition of the examinee, the moving vehicle 10 initializes the flag indicating the standby state, and performs the processing starting from step S120, based on this new movement instruction information.

Besides, if the result of the determination in step S120 is negative, namely, if no new movement instruction information has been acquired, the moving vehicle 10 shifts to step S180, and determines, based on the already acquired movement instruction signal, whether the moving vehicle 10 should stay in the standby state until the hour of departure comes. Then, when the hour of departure comes, the moving vehicle 10 initializes the flag indicating the standby state, shifts to step S170, and starts moving based on the movement instruction signal.

In this manner, according to the present embodiment, the moving vehicle in which the examinee rides can move to the place of exam in accordance with the exam schedule, and the user-friendliness that is offered when the examinee is examined can be improved. In particular, even in the case where the examinee cannot walk by himself or herself, there is no need to transfer the examinee from a bed to a wheelchair or the like, and the burden of assistance can be reduced.

Besides, in the present embodiment, biological information on the examinee is acquired, and the exam schedule is adjusted based on the biological information. Thus, even in the case where the condition of the examinee has changed, the examinee can be examined appropriately and swiftly.

Furthermore, in the present embodiment, the hour of departure of movement is set in accordance with the number of people waiting to be examined. Thus, the waiting time at the destination can be reduced.

### Modification Example

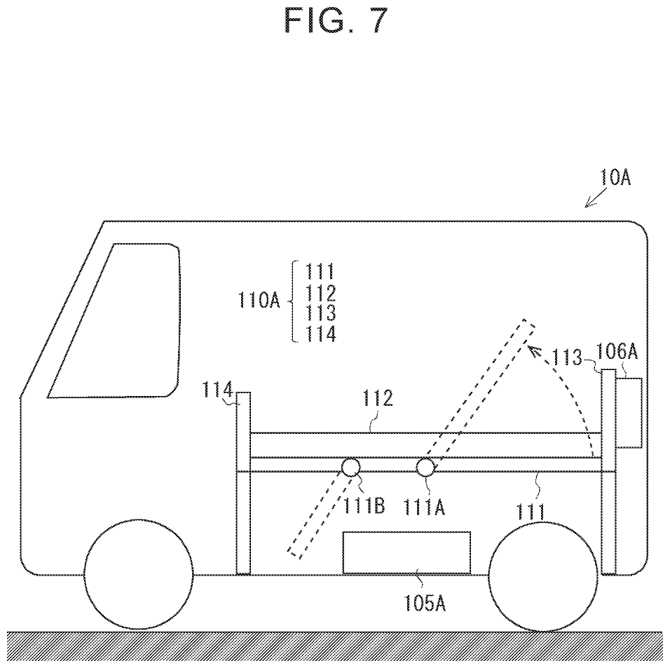

In the foregoing embodiment, the example of the moving vehicle that moves in the hospital has been presented, but the moving vehicle of the disclosure is not limited thereto, but may be an autonomously running vehicle containing a bed. FIG. 7 is a view showing an example of a moving vehicle according to the present modification example.

A moving vehicle 10A shown in FIG. 7 is a vehicle that can autonomously run from a private home of an examinee to a hospital as a place of exam. As shown in FIG. 7, the moving vehicle 10A is equipped with a bed 110A therein. That is, the interior of the moving vehicle 10A serves as a bedroom for the examinee, and the examinee can move to the hospital while lying on the bed in the bedroom.

A drive unit 105A and a control unit 106A of the moving vehicle 10A are identical in function to the drive unit 105 and the control unit 106 in the foregoing embodiment. For example, when the moving vehicle management server 20 transmits a movement instruction signal based on an exam schedule of the examinee, the control unit 106A of the moving vehicle 10A receives this movement instruction signal. Then, the control unit 106A controls the drive unit 105A based on the movement instruction signal, and the moving vehicle 10A autonomously runs to the hospital as the place of exam. It should be noted herein that a known technology can be used as a method of obtaining a running route from the private home of the examinee to the hospital based on positional information on the place of exam indicated by movement instruction information.

Incidentally, when running on a public road, the moving vehicle 10A may have the bed 110A transformed into a sitting mode, and may be able to move with the examinee sitting on the bed 110A.

### Other Embodiments

The above-mentioned embodiment is nothing more than an example, and the disclosure can be carried out after being appropriately altered within such a range as not to depart from the gist thereof. Besides, the processes and means described in the present disclosure can be put into practice freely in combination with one another, as long as no technical contradiction occurs.

Besides, any process described as being performed by a single device may be performed by a plurality of devices in a distributed manner. Alternatively, any process described as being performed by different devices may be performed by a single device. It can be flexibly altered which hardware configuration (which server configuration) should be adopted to realize respective functions of a computer system.

The disclosure can also be realized by supplying a computer with a computer program implementing the functions described in the above-mentioned embodiment, and causing at least one processor belonging to the computer to read out and execute the program. This program may be provided to the computer by a non-transitory computer-readable storage medium that can be connected to a system bus of the computer, or may be provided to the computer via a network. The non-transitory computer-readable storage medium includes, for example, any type of disk such as a magnetic disk (a Floppy® disk, a hard disk drive (HDD), or the like), an optical disk (a CD-ROM, a DVD disk, a Blu-Ray disk, or the like), or the like, a read-only memory (ROM), a random access memory (RAM), an EPROM, an EEPROM, a magnetic card, a flash memory, an optical card, or any type of card that is suited to store electronic commands.

What is claimed is:

1. An information processing device that manages running of a moving vehicle that contains a bed on which an examinee is laid and capable of running autonomously, the information processing device comprising:
a control unit that carries out
acquisition of an exam schedule of the examinee,
generation of a movement instruction signal for moving the moving vehicle to a place of exam, based on the exam schedule, and
transmission of the movement instruction signal to the moving vehicle.

2. The information processing device according to claim 1, wherein
the control unit of the information processing device carries out
acquisition of biological information on the examinee from the moving vehicle, and
adjustment of the exam schedule based on the biological information.

3. The information processing device according to claim 1, wherein
the control unit of the information processing device carries out
acquisition of the number of other examinees waiting to be examined at the place of exam, and
generation of the movement instruction signal based on the exam schedule and the number of waiting examinees.

4. A moving vehicle that contains a bed on which an examinee is laid, and that is able to run autonomously, the moving vehicle comprising:
a drive unit for causing the moving vehicle to run; and
a control unit that controls the drive unit, wherein
the control unit carries out
reception of a movement instruction signal generated based on an exam schedule of the examinee, from another information processing device, and
control of the drive unit based on the movement instruction signal to cause the moving vehicle to autonomously run to a place of exam.

5. An information processing method that is carried out by an information processing device that manages running of a moving vehicle that contains a bed on which an examinee is laid and that is able to run autonomously, the information processing method comprising:
a step of acquiring an exam schedule of the examinee;
a step of generating a movement instruction signal based on the exam schedule; and
a step of transmitting the movement instruction signal to the moving vehicle.
